(19) United States
(12) Patent Application Publication
(10) Pub. No.: US 2018/0114667 A1
(43) Pub. Date: Apr. 26, 2018

(54) ELECTROMAGNET MOUNTING FRAME, ELECTROMAGNET DEVICE, AND PARTICLE BEAM THERAPY SYSTEM

(71) Applicant: Mitsubishi Electric Corporation, Chiyoda-ku, Tokyo (JP)

(72) Inventors: Hiromitsu INOUE, Tokyo (JP); Jun OBATA, Tokyo (JP)

(73) Assignee: Mitsubishi Electric Corporation, Chiyoda-ku, Tokyo (JP)

(21) Appl. No.: 15/572,738

(22) PCT Filed: Sep. 11, 2015
(86) PCT No.: PCT/JP2015/075818

§ 371 Date: Nov. 8, 2017

(51) Int. Cl. H01J 3/38; A61N 5/10

(52) U.S. Cl. CPC H01J 3/38; A61N 5/1077

(57) ABSTRACT

The electromagnet mounting frame is characterized by including: a top plate for supporting the electromagnet; plural legs for sustaining the top plate; and a cable placement member fixed to the plural legs and placed below the top plate; wherein a cable placement portion in which a power cable for the electromagnet is to be placed so as to extend in a traveling direction of the charged particle beam, is formed between the cable placement member and the top plate; and wherein the cable placement portion has a cable placement width (widthwise inter-leg length) that is a length thereof in a direction perpendicular to the traveling direction of the charged particle beam, and that is longer than a width of the electromagnet in the direction perpendicular to the traveling direction of the charged particle beam.

### TECHNICAL FIELD

The present invention relates to an electromagnet mounting frame for supporting an electromagnet that is employed in an accelerator, a beam transport system or the like, used in, for example, research, medical and industrial fields.

### BACKGROUND ART

In general, a particle beam therapy system used for cancer therapy or the like, includes: a beam generation device that generates a charged particle beam; an accelerator that is connected to the beam generation device and accelerates the generated charged particle beam; a beam transport system that transports the charged particle beam emitted from the accelerator after being accelerated thereby up to preset energy; and a particle beam irradiation apparatus that is placed downstream of the beam transport system, for radiating the charged particle beam to an irradiation target. In order to radiate the charged particle beam at an arbitrary angle to the irradiation target, the particle beam irradiation apparatus is placed in a rotary gantry for three-dimensional irradiation.

Charged particles (protons, carbon ions, etc.) accelerated up to high energy in such a manner that the charged particles are circularly accelerated by the accelerator (circular accelerator) such as a synchrotron or the like, are extracted from their circular trajectory, and then the charged particles formed into abeam (referred to also as a charged particle beam or a particle beam) are transported using the beam transport system so as to be applied to a physical experiment in which an intended object is irradiated therewith, or a particle beam therapy such as a cancer therapy or the like. In the cancer therapy using the accelerated charged particles, that is, a so-called particle beam therapy, in order to keep vital organs away or to prevent normal tissues from being affected at the time of the therapy, changing the direction of the irradiation is generally performed. In order to irradiate the patient from an arbitrary direction, the aforementioned particle beam irradiation apparatus placed in the rotary gantry is used.

The accelerator such as a synchrotron or the like, is configured with: a circular acceleration tube in which the charged particle beam circulates; deflection electromagnets and quadrupole electromagnets for controlling the circular trajectory of the charged particle beam; an acceleration cavity for accelerating the charged particle beam using an electric field generated by a high-frequency acceleration voltage; an injection device for introducing the charged particle beam into the acceleration tube; an emission device for extracting the accelerated charged particle beam to the outside; and so on. The deflection electromagnets and the quadrupole electromagnets or the like in the accelerator are supported by mounting frames (for example, Patent Document 1). Further, with respect also to the beam transport system, its deflection electro-magnets and quadrupole electromagnets or the like are supported by mounting frames.

As described above, the accelerator such as a synchrotron or the like, is configured with plural electromagnets, such as the deflection electromagnets and the quadrupole electromagnets, the acceleration cavity, the injection device, the emission device, etc., and in addition, beam measuring instruments, etc. for measuring conditions of the charged particle beam are placed in the accelerator. In an equipment room in which the accelerator and the beam transport system are placed, many cables for instruments that constitute the accelerator and for the beam measuring instruments, etc. for measuring the conditions of the charged particle beam, are placed. In particular, the cables for supplying current (power cables) to the instruments that constitute the accelerator are thick, so that a wide space for placing the cables is required.

For example, in Patent Document 2, a power feeding system for electromagnets in accelerator is described in which an independent power source is provided for each of the different types of electromagnets in terms of excitation current, and cables are laid for each of the types of electromagnets.

### CITATION LIST

### Patent Document

Patent Document 1: Japanese Patent Application Laid-open No. H06-132098 (Paragraph 0065, FIG. 7, FIG. 8)

Patent Document 2: Japanese Patent Application Laid-open No. H07-176400 (Paragraph 0002, FIG. 3)

### SUMMARY OF THE INVENTION

### Problems to be Solved by the Invention

With respect to the power feeding system for electromagnets in accelerator of Patent Document 2, there is no description about mounting frames for supporting the instruments that constitute the accelerator. In general, there are many cables for feeding power (power cables) to the electromagnets, so that, for example, cable racks or the like for storing the power cables are placed in the equipment room in which the accelerator, etc. are placed. The cable racks or the like each have to be placed as it is parallel to the accelerator-constituting instrument supported by the mounting frame, with a given distance therebetween, so that it is required to ensure a space for placing these cable racks or the like, namely, for placing the power cables, in the equipment room. Accordingly, there is a problem that, as the number of the instruments for constituting the accelerator, etc. and the beam measuring instruments, etc., becomes more, the space for placing the power cables becomes larger, so that the equipment room is enlarged.

Further, when the cable rack and the electromagnet are apart from each other, another cable rack for drawing the cable from the above cable rack into the electromagnet will be required. Thus, there is a problem that the space for placing the power cables becomes much larger, so that the equipment room is further enlarged.

The present invention has been made to solve the problems as described above, and an object thereof is to provide an electromagnet mounting frame capable of reducing the space for placing the power cables for electromagnets, in the equipment room in which the electromagnets are placed.

### Means for Solving the Problems

An electromagnet mounting frame of this invention is characterized by comprising: a top plate for supporting an electromagnet; plural legs for sustaining the top plate; and a cable placement member fixed to the plural legs and placed below the top plate; wherein a cable placement portion in which a power cable for the electromagnet is to be placed so as to extend in a traveling direction of the charged particle beam, is formed between the cable placement member and the top plate; and wherein the cable placement portion has a cable placement width that is a length thereof in a direction perpendicular to the traveling direction of the charged particle beam, and that is longer than a width of the electromagnet in the direction perpendicular to the traveling direction of the charged particle beam.

### Effect of the Invention

The electromagnet mounting frame of this invention has the cable placement portion whose cable placement width in the direction perpendicular to the traveling direction of the charged particle beam is longer than the width of the electromagnet. Thus, in the equipment room in which the electromagnet is placed, the power cable can be placed in the cable placement portion, so that it is possible to reduce the space for placing the power cable for the electromagnet.

### BRIEF DESCRIPTION OF THE DRAWINGS

FIG. 1 is a diagram showing an electromagnet mounting frame and an electromagnet device according to Embodiment 1 of the invention.

FIG. 2 is a left side view of FIG. 1.

FIG. 3 is a cross-sectional view of the electromagnet mounting frame in FIG. 1.

FIG. 4 is a diagram showing plural electromagnet devices according to Embodiment 1 of the invention.

FIG. 5 is a left side view of FIG. 4.

FIG. 6 is a diagram showing a particle beam therapy system according to Embodiment 1 of the invention.

FIG. 7 is a diagram showing an electromagnet device and a lifting mechanism according to Embodiment 1 of the invention.

FIG. 8 is a left side view of FIG. 7.

### MODES FOR CARRYING OUT THE INVENTION

### Embodiment 1

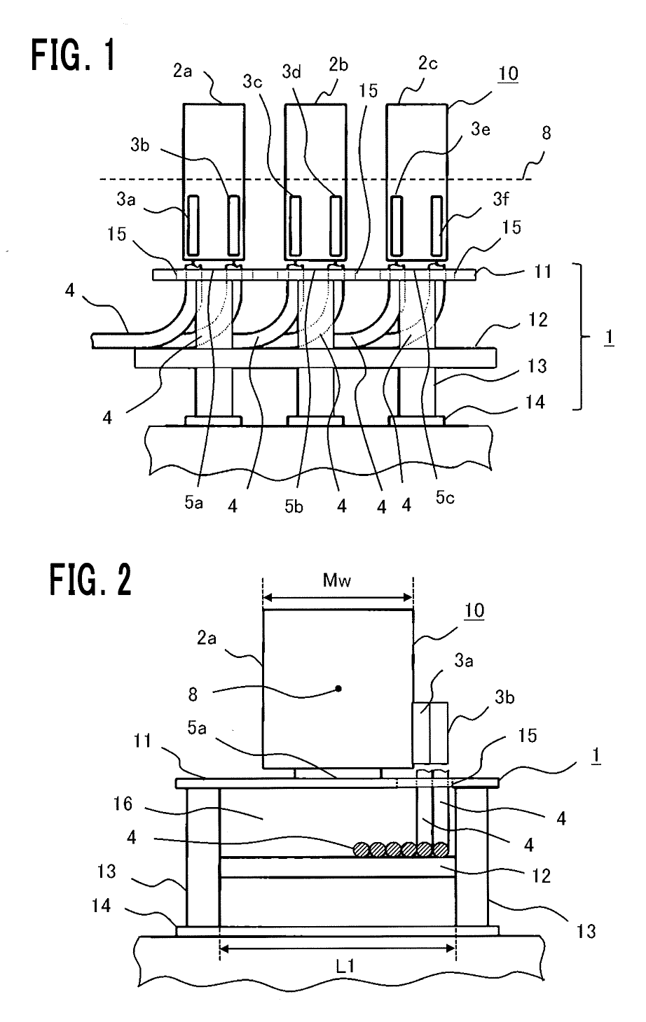

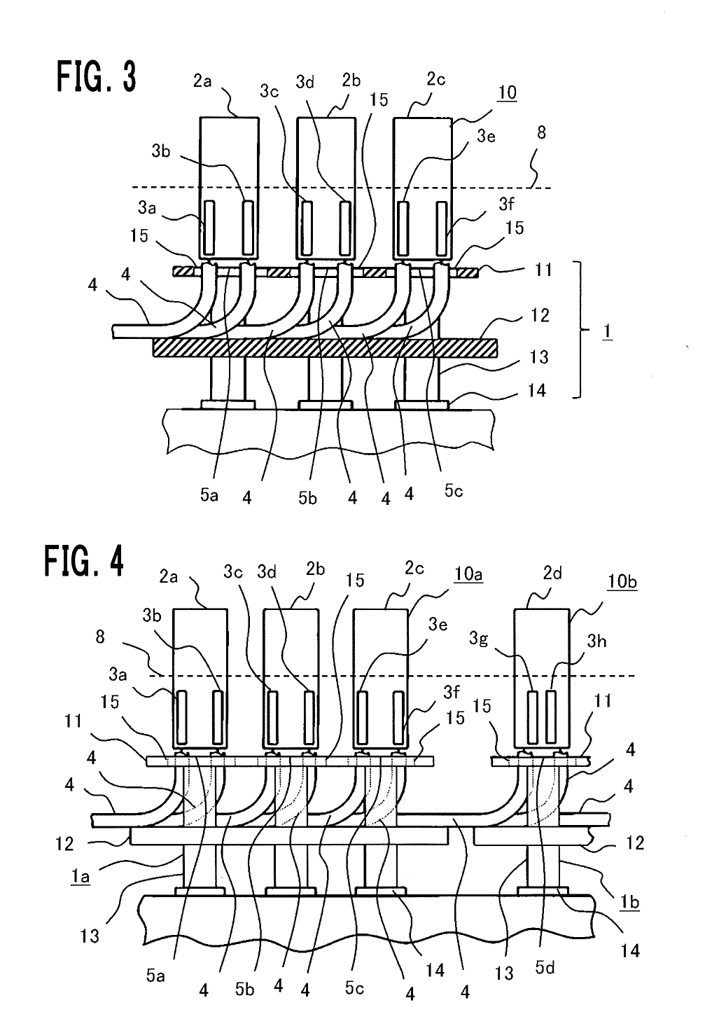

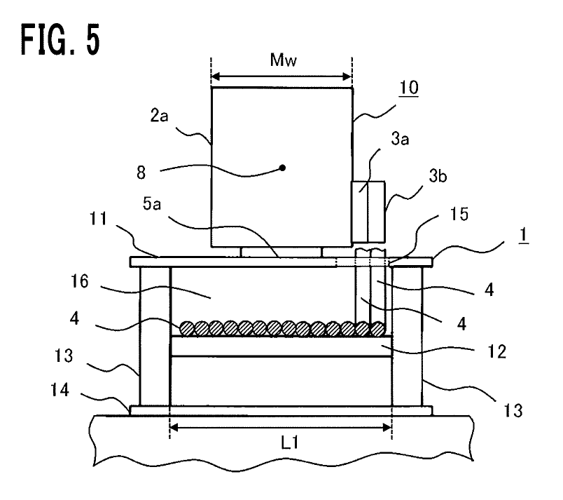

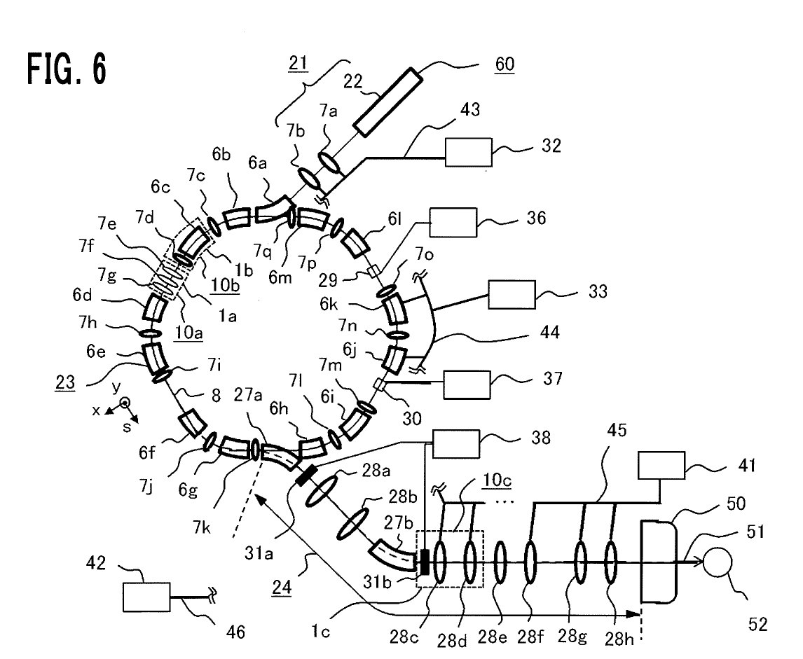

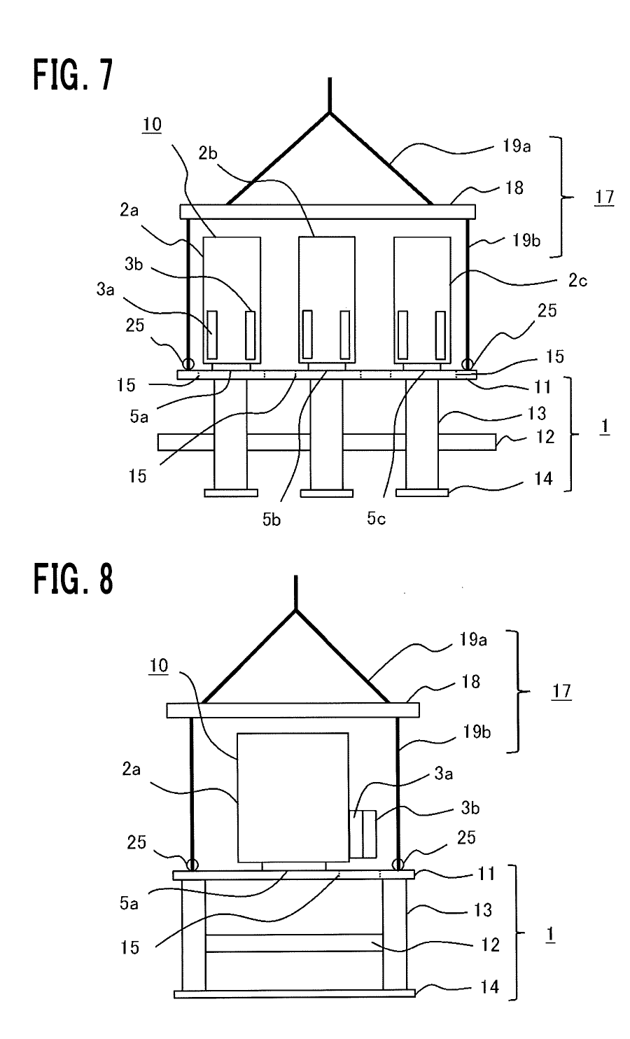

FIG. 1 is a diagram showing an electromagnet mounting frame and an electromagnet device according to Embodiment 1 of the invention, and FIG. 2 is a left side view of FIG. 1. FIG. 3 is a cross-sectional view of the electromagnet mounting frame in FIG. 1, which is a cross-sectional view thereof cut along openings 15 provided in a top plate 11 of the electromagnet mounting frame 1. FIG. 4 is a diagram showing plural electromagnet devices according to Embodiment 1 of the invention, and FIG. 5 is a left side view of FIG. 4. FIG. 6 is a diagram showing a particle beam therapy system according to Embodiment 1 of the invention. FIG. 7 is a diagram showing an electromagnet device and a lifting mechanism according to Embodiment 1 of the invention, and FIG. 8 is a left side view of FIG. 7. An electromagnet device 10 includes plural electromagnets 2a, 2b, 2c for causing magnetic fields to act on a charged particle beam, and the electromagnet mounting frame 1 for supporting these electromagnets 2a, 2b, 2c. The electromagnet mounting frame 1 includes: the top plate 11 on which the plural electromagnets 2a, 2b, 2c are placed; a power-cable placement member 12 on which plural power cables 4 to be connected to the plural electromagnets 2a, 2b, 2c are laid; plural legs 13 for sustaining the top plate 11; and plural bottom plates 14 provided at bottom portions of the legs 13.

The legs 13 of the electromagnet mounting frame 1 are poles for supporting the electromagnets, the number of which may vary depending on the sizes, weights and mounted number of the electromagnets, but is generally four to six. Further, as the material of the electromagnet mounting frame 1, iron is generally used. At the top plate 11, plural electromagnet support portions 5a, 5b, 5c for supporting the plural electromagnets 2a, 2b, 2c, and the openings 15 through which the power cables 4 to be connected to the plural electromagnets 2a, 2b, 2c pass, are provided. The electromagnets 2a, 2b, 2c are mounted on the electromagnet support portions 5a, 5b, 5c, respectively. In FIG. 1 to FIG. 3, such an example is shown in which three openings 15 are provided so as to correspond to the three electromagnets 2a, 2b, 2c to be mounted. To power-cable connection terminals 3a, 3b of the electromagnet 2a, the power cables 4 passing through the opening 15 are connected, respectively. Likewise, to power-cable connection terminals 3c, 3d of the electromagnet 2b, the power cables 4 passing through the opening 15 are connected, respectively. To power-cable connection terminals 3e, 3f of the electromagnet 2c, the power cables 4 passing through the opening 15 are connected, respectively. Note that, in FIG. 1 to FIG. 5, connection portions between core wires of the power cables 4 and the power-cable connection terminals of the electromagnets are omitted from illustration.

The power cables 4 for the electromagnets 2a, 2b, 2c mounted on the electromagnet mounting frame 1 are placed in a power-cable placement portion 16 (see, FIG. 2). The power-cable placement portion 16 is a region placed on the inner side of the legs 13 and between the power-cable placement member 12 and the top plate 11. In FIG. 1 to FIG. 3, such an example is shown in which six power cables 4 are laid on the power-cable placement member 12. As shown in FIG. 4 and FIG. 5, in the power-cable placement portion 16, the power cables 4 for an electromagnet mounted on another electromagnet mounting frame 1 may also be placed. As a space (volume) of the power-cable placement portion 16, such a space is ensured for every electromagnet mounting frame 1 that is matched to the number of the power cables 4 to be connected to the electromagnets for constituting the accelerator, etc. Namely, when the number of the power cables 4 to be placed in the power-cable placement portion 16 is large, the length (inter-leg length) between the legs 13 placed in a direction perpendicular to the extending direction of the power cables 4 is made longer than otherwise. Further, when the power cables 4 are to be placed in a mutually overlapping manner, the interval between the power-cable placement member 12 and the top plate 11 is made longer than otherwise. In the electromagnet mounting frame 1 closer to the electromagnet power source, the power cables 4 are placed more, so that the power-cable placement portion 16 in that electromagnet mounting frame 1 is made larger (its volume is increased). In the electromagnet mounting frame 1 farther from the electromagnet power source, the number of the power cables 4 to be placed is less than that in another electromagnet mounting frame closer to the electromagnet power source, so that the power-cable placement portion 16 in that electromagnet mounting frame 1 may be made smaller (its volume may be decreased).

In FIG. 6, a particle beam therapy system 60 is shown which is provided with an accelerator 23 and a beam transport system that have plural electromagnets. In FIG. 6, electromagnet devices 10a, 10b that are placed adjacent to each other are shown. The electromagnets mounted in the electromagnet devices 10a, 10b are adjusted in their attitudes and heights, namely, their placement positions, in order that a charged particle beam 51 is transported along abeam line 8. In the electromagnet device 10 shown in FIG. 1 and FIG. 2, the electromagnets 2a, 2b, 2c are placed on the electromagnet mounting frame 1 so that the beam line 8 of the accelerator 23, the beam transport system 24, etc. passes through the center portions of the electromagnets 2a, 2b, 2c. Description on the particle beam therapy system 60 will be made later. FIG. 4 and FIG. 5 show an example in which, for example, the plural power cables 4 are laid in the electromagnet mounting frames 1a, 1b of the electromagnet devices 10a, 10b that are placed adjacent to each other so as to contain the beam line 8 along which the charged particle beam 51 accelerated by the accelerator passes. The electromagnet mounting frames 1a, 1b are placed below the beam line 8. In FIG. 4, an electromagnet 2d is mounted on an electromagnet support portion 5d, and the cables 4 passing through the opening 15 are connected to respective power-cable connection terminals 3g, 3h of the electromagnet 2d. In FIG. 5, fourteen power cables 4 are shown. Although the number of the power cables 4 for the electromagnets 2a, 2b, 2c, 2d illustrated in FIG. 5 is eight, the power cables 4 for other electromagnets are also shown therein.

In FIG. 2 and FIG. 5, in order to lay the power cables 4 for such plural electromagnets that constitute the accelerator, etc., the interval of the legs 13 (inter-leg interval) of the electromagnetic mounting frame 1 is enlarged, namely, as described previously, the inter-leg length between the legs 13 placed in a direction perpendicular to the extending direction of the power cables 4, that is, a widthwise inter-leg length L1, is made longer. Note that the widthwise inter-leg length L1 is also a cable placement width that is a length of the power-cable placement portion 16 in a direction perpendicular to the traveling direction of the charged particle beam. In general, in order to merely sustain the electromagnets, the inter-leg length between the legs 13 placed in a direction perpendicular to the extending direction of the power cables 4, may be shorter than that shown in FIG. 2 or FIG. 5. However, in this embodiment, the widthwise inter-leg length L1 is made so long, thus enlarging the power-cable placement portion 16, so that the power cables 4 whose number is more than the number of power cables to be connected to the electromagnets mounted on one electromagnet mounting frame 1, can be laid in that portion. The widthwise inter-leg length L1 between the legs 13 of the electromagnet mounting frame 1 is longer than a width Mw in a direction perpendicular to the beam line 8, of the electromagnet mounted on the electromagnet mounting frame 1. For example, the widthwise inter-leg length L1 is 1.5 times or more the width Mw of the electromagnet.

Using FIG. 6, the particle beam therapy system 60 will be described which is provided with the accelerator 23 and the beam transport system 24 that have the plural electromagnets. The particle beam therapy system 60 includes an injection system 21, the accelerator 23, the beam transport system 24 and a particle beam irradiation apparatus 50. The injection system 21 has an injection device 22 and quadrupole electromagnets 7a, 7b. The accelerator 23 includes: plural deflection electromagnets 6a, 6b, 6c, 6d, 6e, 6f, 6g, 6h, 6i, 6j, 6k, 6l, 6m; plural quadrupole electromagnets 7c, 7d, 7e, 7f, 7g, 7h, 7i, 7j, 7k, 7l, 7m, 7n, 7o, 7p, 7q; an acceleration cavity 29; an x-direction kick electrode 30; a high-frequency acceleration power source 36; a high-frequency kick power source 37; an electromagnet power source 33 for supplying current to the plural deflection electromagnets; and an electromagnet power source 32 for supplying current to the plural quadrupole electromagnets. The beam transport system 24 includes: plural deflection electromagnets 27a, 27b; plural quadrupole electromagnets 28a, 28b, 28c, 28d, 28e, 28f, 28g, 28h; beam profile monitors 31a, 31b; a beam analysis device 38; an electromagnet power source 41 for supplying current to the plural quadrupole electromagnets; and an electromagnet power source 42 for supplying current to the plural deflection electromagnets. A beam transport tube (not shown) is placed from the injection device 22 to the particle beam therapy system 50, so as to enclose the beam line 8. For the quadrupole electromagnets in the injection system 21 and the accelerator 23, numeral 7 is used collectively, and numerals 7a to 7q are used when they are to be described distinctively. For the deflection electromagnets in the accelerator 23, numeral 6 is used collectively, and numerals 6a to 6m are used when they are to be described distinctively. For the quadrupole electromagnets in the beam transport system 24, numeral 28 is used collectively, and numerals 28a to 28h are used when they are to be described distinctively. For the deflection electromagnets in the beam transport system 24, numeral 27 is used collectively, and numerals 27a, 27b are used when they are to be described distinctively.

The quadrupole electromagnets 7 in the injection system 21 and the accelerator 23 are connected using a power cable 43 to the electromagnet power source 32. The deflection electromagnets 6 in the accelerator 23 are connected using a power cable 44 to the electromagnet power source 33. The quadrupole electromagnets 28 in the beam transport system 24 are connected using a power cable to the electromagnet power source 41. The deflection electromagnets 27 in the beam transport system 24 are connected using a power cable 46 to the electromagnet power source 42. In FIG. 6, three electromagnet devices 10a, 10b, 10c and their respective electromagnet mounting frames 1a, 1b, 1c are shown. On the electromagnet mounting frame 1a, three quadrupole electromagnets 7e, 7f, 7g are mounted, and on the electromagnet mounting frame 1b, the quadrupole electromagnet 7d and the deflection electromagnet 6c are mounted. On the electromagnet mounting frame 1c, two quadrupole electromagnets 28c, 28d and the beam profile monitor 31b are mounted. Note that, although the electromagnets that constitute the injection system 21, the accelerator 23 and the beam transport system 24 are mounted on the electromagnet mounting frames 1, only three electromagnet mounting frames 1a, 1b, 1c are shown in FIG. 6.

The deflection electromagnets 6, 27 in the particle beam therapy system 60 each deflect the charged particle beam 51, and the quadrupole electromagnets 7, 28 in the particle beam therapy system 60 each converge or diverge the charged particle beam 51. In the beam coordinate system for the charged particle beam 51, an axis in the traveling direction (s-direction) of the charged particle beam 51 is referred to as an s-axis, an axis in an x-direction that is a direction perpendicular to the s-axis and outwardly extending in the plane of the circular trajectory in the accelerator 23 is referred to as an x-axis, and an axis in a y-direction that is perpendicular to the s-axis and the x-axis is referred to as a y-axis. The acceleration cavity 29 accelerates the charged particle beam 51 circulating in the accelerator 23. The x-direction kick electrode 30 is an electrode for ejecting the charged particle beam 51 outwardly (in the x-direction) from its circulating direction with an electric field so as to be emitted into the beam transport system 24. The beam profile monitors 31a, 31b detect beam profile data for calculating the beam position, the beam size, etc. of the charged particle beam 51. The beam analysis device 38 acquires the profile data detected by the beam profile monitors 31a, 31b, to thereby analyze the beam position. The beam transport system 24 transports the charged particle beam 51 to the particle beam irradiation apparatus 50. The particle beam irradiation apparatus 50 radiates the charged particle beam 51 to the irradiation target 52.

The charged particle beam 51 that is a particle beam, such as proton beam or the like, generated by an ion source in the injection device 22, is accelerated by a pre-accelerator in the injection device 22, and then the charged particle beam 51 is injected while being converged or diverged by the quadrupole electromagnets 7a, 7b, into the accelerator 23. Here, description will be made citing a synchrotron as an example of the accelerator 23. The charged particle beam 51 is accelerated up to given energy. The charged particle beam 51 enters from the deflection electromagnet 27a placed in the accelerator 23 into the beam transport system 24, so that it is transported to the particle beam irradiation apparatus 50 and is then radiated by the particle beam irradiation apparatus 50 to a diseased site that is the irradiation target 52 in the patient. The particle beam irradiation apparatus 50 radiates the charged particle beam 51 to the irradiation target 52 while enlarging the beam or scanning the beam so that the beam forms an intended irradiation field.

The electromagnet mounting frames 1a, 1b shown in FIG. 6 have a positional relationship that is similar to that of the electromagnet mounting frames 1a, 1b shown in FIG. 4. The electromagnets 2a, 2b, 2c, 2d shown in FIG. 4 correspond to the quadrupole electromagnets 7g, 7f, 7e, 7d in FIG. 6. An electromagnet to be mounted on the electromagnet mounting frame 1 of this embodiment is not limited to a quadrupole electromagnet, and thus a dipole electromagnet such as a deflection electromagnet, or an electromagnet of another type such as a sextupole electromagnet or the like, may be mounted thereon (see, the electromagnet mounting frame 1b in FIG. 6). Further, on the electromagnet mounting frame 1 of this embodiment, other than the electromagnet, a beam measuring instrument such as a beam profile monitor, a beam position monitor or the like, may be mounted (see, the electromagnet mounting frame 1c in FIG. 6). Note that, on the electromagnet mounting frame 1 of this embodiment, only the beam measuring instrument such as a beam profile monitor, a beam position monitor or the like, may be mounted. Further, there is no limit on the numbers of the electromagnets and the beam measuring instruments to be mounted on the electromagnet mounting frame 1. When the electromagnet is heavy, one electromagnet is mounted per one electromagnet mounting frame 1. The electromagnet mounting frame 1 of Embodiment 1 can be manufactured using design parameters, such as the size thereof, the shape of the top plate, the number of mounted electromagnets, and the like, which are chosen flexibly in conformity with the individual particle beam therapy system 60 provided with plural electromagnets. The shape of the top plate may be bent along the beam line 8 as shown, for example, by the electromagnet mounting frame 1b in FIG. 6.

According to the electromagnet mounting frame 1 of Embodiment 1, the widthwise inter-leg length L1 between the legs 13 is longer than the width Mw in the direction perpendicular to the beam line 8, of the electromagnet mounted on the electromagnet mounting frame 1, so that the power cables 4 for the plural electromagnets can be laid in the power-cable placement portion 16. Accordingly, as compared with the conventional case where a large power-cable placement space for placing cable racks that store the power cables is required, the electromagnet mounting frame 1 of Embodiment 1 makes it possible to reduce such a power-cable placement space provided for placing the cable racks. Further, since the electromagnet mounting frame 1 of Embodiment 1 makes it possible to reduce, as compared with the conventional case, such a power-cable placement space provided for placing the cable racks, the equipment room in which the accelerator, etc. are placed can be made smaller than that in the conventional case, namely, it is possible to accomplish more efficient placement space for the instruments in the equipment room.

In the electromagnet mounting device 10 of Embodiment 1, the electromagnet mounting frame 1 and the electromagnets 2a, 2b, 2c are unified together. Thus, when wire connectors 25 such as lifting hooks or the like, are provided on the electromagnet mounting frame 1, it is possible, as shown in FIG. 7 and FIG. 8, to convey using a lifting beam 18 and lifting wires 19a, 19b, the electromagnet device 10 in a state in which the electromagnets 2a, 2b, 2c are mounted on the electromagnet mounting frame 1. A lifting mechanism 17 for lifting the electromagnet device 10 includes the lifting beam 18 and the lifting wires 19a, 19b. When the lifting wires 19b of the lifting mechanism 17 are connected to the wire connectors 25 of the electromagnet mounting device 10 of Embodiment 1, it is possible to carry the electromagnet device 10 in which the electromagnet mounting frame 1 and the electromagnets 2a, 2b, 2c are unified together, in a cargo container or the like. The cargo container or the like in which the electromagnet device 10 is stored will be conveyed to the placement site of the electromagnet device 10, using a conveyance means, such as a truck, a train or the like.

According to the electromagnet device 10 of Embodiment 1, since the electromagnet mounting frame 1 and the electromagnets 2a, 2b, 2c are unified together, as compared with an electromagnet mounting device in which they are not unified, an on-site work for mounting the electromagnets 2a, 2b, 2c on the electromagnet mounting frame 1 can be eliminated, so that it is possible to simplify the on-site construction work and/or to shorten the time therefor. According to the electromagnet device 10 of Embodiment 1, it is allowable, in its manufacturing facility, to precisely perform position adjustment for accommodating the plural electromagnets 2a, 2b, 2c mounted on the electromagnet mounting frame 1, to the beam line 8 at the placement site. This allows on-site adjustment for accommodating the positions of the plural electromagnets 2a, 2b, 2c to the beam line 8, to be just fine adjustment, so that a work for the adjustment for accommodating them to the beam line 8 can also be shortened. According to the electromagnet device 10 of Embodiment 1, the more the number of the electromagnets to be mounted on one electromagnet mounting frame 1 becomes, the more simplified the on-site construction work can be and the more shortened the time for that work can be.

As described above, the electromagnet mounting frame 1 of Embodiment 1 is an electromagnet mounting frame 1 for supporting the electromagnet 2a that causes a magnetic field to act on the charged particle beam 51, said electromagnet mounting frame characterized by comprising: the top plate 11 for supporting the electromagnet 2a; the plural legs 13 for sustaining the top plate 11; and a cable placement member (power-cable placement member 12) fixed to the plural legs 13 and placed below the top plate 11; wherein a cable placement portion (power-cable placement portion 16) in which the power cables 4 for the electromagnet 2a are to be placed so as to extend in the traveling direction of the charged particle beam 51, is formed between the cable placement member (power-cable placement member 12) and the top plate 11. In the electromagnet mounting frame 1, the cable placement portion (power-cable placement portion 16) is characterized by having a cable placement width (widthwise inter-leg length L1) that is a length thereof in a direction perpendicular to the traveling direction of the charged particle beam 51, and that is longer than the width Mw of the electromagnet 2a in the direction perpendicular to the traveling direction of the charged particle beam 51. According to these characteristics, the electromagnet mounting frame 1 of Embodiment 1 has the cable placement portion (power-cable placement portion 16) whose cable placement width (widthwise inter-leg length L1) that is perpendicular to the traveling direction of the charged particle beam 51, is longer than the width Mw of the electromagnet 2a. Thus, in the equipment room in which the electromagnet 2a is placed, it is possible to reduce the space for placing the power cables for the electromagnet 2a.

Further, the electromagnet device 10 of Embodiment 1 includes the electromagnet mounting frame 1 and at least one of the electromagnets 2a, 2b, 2c that is mounted on the top plate 11 of the electromagnet mounting frame 1. The electromagnet mounting frame 1 in the electromagnet device 10 of Embodiment 1 is an electromagnet mounting frame 1 for supporting the electromagnet 2a, 2b, 2c that causes a magnetic field to act on the charged particle beam 51, said electromagnet mounting frame characterized by comprising: the top plate 11 for supporting the electromagnet 2a, 2b, 2c; the plural legs 13 for sustaining the top plate 11; and a cable placement member (power-cable placement member 12) fixed to the plural legs 13 and placed below the top plate 11; wherein a cable placement portion (power-cable placement portion 16) in which the power cables 4 for the electromagnet 2a, 2b, 2c are to be placed so as to extend in the traveling direction of the charged particle beam 51, is formed between the cable placement member (power-cable placement member 12) and the top plate 11 (characteristic 1). In the electromagnet mounting frame 1, the cable placement portion (power-cable placement portion 16) is characterized by having a cable placement width (widthwise inter-leg length L1) that is a length thereof in a direction perpendicular to the traveling direction of the charged particle beam 51, and that is longer than the width Mw of the electromagnet 2a, 2b, 2c in the direction perpendicular to the traveling direction of the charged particle beam 51 (characteristic 2). According to these characteristics, the electromagnet device 10 of Embodiment 1 makes it possible to reduce the space for placing the power cables for the electromagnet 2a, 2b, 2c, in the equipment room in which the electromagnet 2a, 2b, 2c is placed, and to simplify the on-site construction work and/or to shorten the time therefor.

The particle beam therapy system 60 of Embodiment 1 comprises: the injection system 21 in which the charged particle beam 51 is generated; the accelerator 23 for accelerating the charged particle beam 51 injected thereto from the injection system 21; the beam transport system 24 for transporting the charged particle beam accelerated by the accelerator 23; and the particle beam irradiation apparatus 50 for radiating the charged particle beam 51 transported by the beam transport system 24, to the irradiation target 52; wherein both or either one of the accelerator 23 and the beam transport system 24 is provided with the plural electromagnet devices 10 in each which the electromagnet 2a, 2b, 2c is mounted. The particle beam therapy system 60 of Embodiment 1 is characterized in that: in the cable placement portion (power-cable placement portion 16) of at least one of the electromagnet devices 10, together with the power cables 4 connected to the electromagnet 2a, 2b, 2c in said at least one electromagnet device 10, the power cables 4 to be connected to the electromagnet 2a, 2b, 2c in the other electromagnet device 10 are placed; the electromagnet devices 10 each comprise the electromagnet mounting frame 1 and at least one of the electromagnets 2a, 2b, 2c mounted on the top plate 11 of the electromagnet mounting frame 1; and the electromagnet devices 10 each have the characteristic 1 and the characteristic 2. According to these characteristics, the space for placing the power cables for the electromagnet 2a, 2b, 2c can be reduced in the equipment room in which the electromagnet 2a, 2b, 2c is placed, and the on-site construction work can be simplified and/or the time for that work can be shortened.

It is noted that the electromagnet mounting frame 1 may be applied not only to a synchrotron, but also to a commonly-used accelerator, such as a linear accelerator, a cyclotron or the like. Further, combination of respective embodiments and an appropriate modification/omission in the embodiments may be made in the present invention without departing from the scope of the invention.

### DESCRIPTION OF REFERENCE NUMERALS AND SIGNS

1, 1a, 1b, 1c: electromagnet mounting frame,
2a, 2b, 2c, 2d: electromagnet,
4: power cable,
5a, 5b, 5c, 5d: electromagnet support portion,
6, 6a, 6b, 6c, 6d, 6e, 6f, 6g, 6h, 6i, 6j, 6k, 6l, 6m: deflection electromagnet,
7, 7a, 7b, 7c, 7d, 7e, 7f, 7g, 7h, 7i, 7j, 7k, 7l, 7m, 7n, 7o, 7p, 7q: quadrupole electromagnet,
10, 10a, 10b, 10c: electromagnet device,
11: top plate,
12: power-cable placement member,
13: leg,
15: opening,
16: power-cable placement portion,
19a, 19b: lifting wire,
21: injection system,
23: accelerator,
24: beam transport system,
25: wire connector,
27, 27a, 27b: deflection electromagnet,
28, 28a, 28b, 28c, 28d, 28e, 28f, 28g, 28h: quadrupole electromagnet,
31a, 31b: beam profile monitor,
43: power cable,
44: power cable,
45: power cable,
46: power cable,
50: particle beam irradiation apparatus,
51: charged particle beam,
52: irradiation target,
60: particle beam therapy system,
Mw: width of electromagnet,
L1: widthwise inter-leg length (cable placement width).

1. An electromagnet mounting frame for supporting an electromagnet that causes a magnetic field to act on a charged particle beam, comprising:
a top plate for supporting the electromagnet;
plural legs for sustaining the top plate; and
a cable placement member fixed to the plural legs and placed below the top plate;
wherein a cable placement portion in which a power cable for the electromagnet is to be placed so as to extend in a traveling direction of the charged particle beam, is formed between the cable placement member and the top plate; and
wherein the cable placement portion has a cable placement width that is a length thereof in a direction perpendicular to the traveling direction of the charged particle beam, and that is longer than a width of the electromagnet in the direction perpendicular to the traveling direction of the charged particle beam.

2. The electromagnet mounting frame of claim 1, wherein the top plate has an opening that allows the power cable for the electromagnet to pass therethrough from the cable placement portion to the upper side of the top plate.

3. The electromagnet mounting frame of claim 1, wherein the top plate has a wire connector to which a wire for lifting the electromagnet mounting frame is to be connected.

4. The electromagnet mounting frame of claim 1, wherein the cable placement portion has the cable placement width that is 1.5 times or more the width of the electromagnet in the direction perpendicular to the traveling direction of the charged particle beam.

5. The electromagnet mounting frame of claim 1, wherein the top plate has plural electromagnet support portions for supporting plural electromagnets each being said electromagnet that are to be placed in the traveling direction of the charged particle beam.

6. An electromagnet device comprising:
the electromagnet mounting frame of claim 1; and
the electromagnet mounted on the top plate of the electromagnet mounting frame.

7. An electromagnet device comprising:
the electromagnet mounting frame of claim 5; and
the plural electromagnets mounted on the electromagnet support portions of the top plate in the electromagnet mounting frame.

8. A particle beam therapy system which comprises: an injection system in which a charged particle beam is generated; an accelerator for accelerating the charged particle beam injected thereto from the injection system; a beam transport system for transporting the charged particle beam accelerated by the accelerator; and a particle beam irradiation apparatus for radiating the charged particle beam transported by the beam transport system, to an irradiation target;
wherein both or either one of the accelerator and the beam transport system is provided with plural electromagnet devices each being said electromagnet device of claim 6 in which the electromagnet(s) is/are mounted; and
wherein, in the cable placement portion of at least one of the electromagnet devices, together with the power cable connected to the electromagnet in said at least one electromagnet device, the power cable to be connected to the electromagnet in the other electromagnet device is placed.

9. The particle beam therapy system of claim 8, wherein, in at least one of the electromagnet devices, a beam measuring instrument for measuring a condition of the charged particle beam is mounted on the top plate.

10. The electromagnet mounting frame of claim 2, wherein the top plate has a wire connector to which a wire for lifting the electromagnet mounting frame is to be connected.

11. The electromagnet mounting frame of claim 2, wherein the cable placement portion has the cable placement width that is 1.5 times or more the width of the electromagnet in the direction perpendicular to the traveling direction of the charged particle beam.

12. The electromagnet mounting frame of claim 3, wherein the cable placement portion has the cable placement width that is 1.5 times or more the width of the electromagnet in the direction perpendicular to the traveling direction of the charged particle beam.

13. The electromagnet mounting frame of claim 2, wherein the top plate has plural electromagnet support portions for supporting plural electromagnets each being said electromagnet that are to be placed in the traveling direction of the charged particle beam.

14. The electromagnet mounting frame of claim 3, wherein the top plate has plural electromagnet support portions for supporting plural electromagnets each being said electromagnet that are to be placed in the traveling direction of the charged particle beam.

15. The electromagnet mounting frame of claim 4, wherein the top plate has plural electromagnet support portions for supporting plural electromagnets each being said electromagnet that are to be placed in the traveling direction of the charged particle beam.

16. An electromagnet device comprising:
the electromagnet mounting frame of claim 2; and
the electromagnet mounted on the top plate of the electromagnet mounting frame.

17. An electromagnet device comprising:
the electromagnet mounting frame of claim 3; and
the electromagnet mounted on the top plate of the electromagnet mounting frame.

18. An electromagnet device comprising:
the electromagnet mounting frame of claim 4; and
the electromagnet mounted on the top plate of the electromagnet mounting frame.

19. A particle beam therapy system which comprises: an injection system in which a charged particle beam is generated; an accelerator for accelerating the charged particle beam injected thereto from the injection system; a beam transport system for transporting the charged particle beam accelerated by the accelerator; and a particle beam irradiation apparatus for radiating the charged particle beam transported by the beam transport system, to an irradiation target;
wherein both or either one of the accelerator and the beam transport system is provided with plural electromagnet devices each being said electromagnet device of claim 7 in which the electromagnet(s) is/are mounted; and
wherein, in the cable placement portion of at least one of the electromagnet devices, together with the power cable connected to the electromagnet in said at least one electromagnet device, the power cable to be connected to the electromagnet in the other electromagnet device is placed.
